(19) United States
(12) Patent Application Publication
(10) Pub. No.: US 2020/0352787 A1
(43) Pub. Date: Nov. 12, 2020

(54) Light-Shielding Body Structure And Novel Eyeshade Structure

(71) Applicant: Xiamen Sunnypet Products Co., Ltd., Xiamen, Fujian (CN)

(72) Inventor: Kongzhi LU, Xiamen, Fujian (CN)

(73) Assignee: Xiamen Sunnypet Products Co., Ltd., Xiamen, Fujian (CN)

(21) Appl. No.: 16/758,845

(22) PCT Filed: Sep. 26, 2018
(86) PCT No.: PCT/CN2018/107465

§ 371 Date: Apr. 23, 2020

(30) Foreign Application Priority Data
Sep. 26, 2018 (CN) PCT/CN2018/107465

(51) Int. Cl. A61F 9/04; A61F 9/06

(52) U.S. Cl. CPC A61F 9/045; A61F 9/06

(57) ABSTRACT

Present invention teaches a light-blocking (shielding) structure, as well as a novel eye mask, that has a base with two light-shielding bodies, with additional area supports, to effectively blocks light from the gaps around the mask, pad or patches, by using flexible materials that can contour or conform to the shape of a human nose, thus creating a better “darkness” effect to enhance quality sleep experience.

### CLAIM OF PCT APPLICATION, 35 U.S.C. § 371

The present invention is a national stage entry from a PCT application, pursuant to the provision of 35 U.S.C. section 371 et seq., based upon the application filed by the same inventor in China, having PCT application number PCT/CN2018/107465 filed on Sep. 26, 2018, which claims the priority filing date of Oct. 24, 2017 of Chinese Application No.: 201710999194.4.

### FIELD AND BACKGROUND OF THE INVENTION

In today's high pressure world, people are not getting quality sleep. There is certainly abundant, and even excessive, consumption in people's daily lives, but the lack of sleep, quality sleep particularly, is simply not compensated by heavier consumption. As a result, more and more people are paying attention to the issue of enhancing quality of sleep.

One of the solutions to getting quality sleep is by wearing a face mask that covers both eyes, or some form of eye patch(s) to shield off light when sleeping, or for short nap either during a normal work day, during commute, or any ad hoc down time.

Blocking off light will create a setting that is closer to the night environment, which is more conducive to getting quality sleep.

Although face masks, or eye pads/patches, are generally available for the purpose stated herein, the shape, design or material are not ideal, generally causing at least a gap to exist at a spot near a human's nose ridge. As such, the “darkness” effect intended by wearing face mask or eye patches is not good, and the assistance to help achieve quality sleep is rather limited.

A Chinese patent, CN 202128604 U, published an eye pad, consisting of a light-shielding unit and a wear-band connected to the two sides of the eye pad. The light-shielding unit's inside surface, at the lower edge, has a soft light-blocking piece that can be pressed closer to the sides of a nose ridge. The problem of the gap becomes smaller, but is still there.

Other available face masks and eye patches/pads also come up with some minor improvements to add blocking materials under the light-shielding units and around the nose, however, the problem of the gap remains.

An important reason for the problem is that human faces have different curvatures, and even for the eye pads/patches or face masks worn by the same individual, the facial curvature will change during a normal sleep (or nap) session due to people's natural turning or rolling movements.

As such, the light-shielding functionality that can be fitted snugly to human faces and be flexible in response to external forces or pressure is needed to help with the reduction of the “gap” and to enhance the “darkness” that is conducive for quality sleep.

### SUMMARY OF THE INVENTION

To solve the light leakage (due to the existence of a gap) issue, the present invention disclosed and claimed a light-shielding body structure and a novel eye mask (or eye shade) structure employing the light-shielding body structure, to create a “darkness” setting conducive to quality sleep, or nap.

The embodiment of the light-shielding body structure of present invention is primarily comprised of a base and two light-shielding bodies that can flexibly change shape when pressed upon.

The two light-shielding bodies are symmetrically placed on two sides of said base.

The two light-shielding bodies are made to be arc-shaped and bent upwards relative to said base, forming an adjustment area that can be fitted to a human nose wing portion.

In the disclosure herein, the term “nose wing(s)” will be used to denote the two sides of a nose ridge, for purpose of describing the light-shielding body structure's fitting to the nose portion of a human face.

Each of the two light-shielding bodies further consists of a first support part that is meant to stay close to a human nose wing, and a second support part that is meant to stay close to a human face, with the height of projection of the first support part gradually tapers down towards the second support part.

Each of the light-shielding body's arc-shape is further made to form a ring shape and are connected to the two sides of the base.

Each of the light-shielding bodies further consists of a piece of cloth and a folded light-shielding sheet wrapped inside the cloth.

A novel eye mask (eye shade) structure of present invention is formed by combining the light-shielding body structure described above with a mask body and two tie bands on the two sides of the mask body.

Such a novel eye mask structure is good for placing over both eyes to block off light.

The novel eye mask structure further has a base (part of the light-shielding body structure) that is substantially the same as the shape of the mask body and the base is connected to inner portion of the mask body.

The novel eye mask structure further has its base contoured to the shape of a human nose, situated in a central part on the inside of the mask body, and the two light-shielding bodies are connected to the two sides of the base and the peripheral edge on the inside of the mask body.

The novel eye mask structure has its first light-shielding body further connected to the left side and the upper-left part of the mask body's inside surface and its second light-shielding body further connected to the right side and upper-right part of the mask body's inside surface.

The novel eye mask structure further comprises of an outer shell and an inner contact piece, incorporating to the light-shielding body structure described herein. The light-shielding body of the novel mask structure is connected to the inner contact piece. Said inner contact piece further consists of a first contact piece and a second contact piece, so that the left side of the base is connected to the first contact piece via the first light-shielding body, and the right side of the base is connected to the second contact piece via the second light-shielding bod.

Given the embodiment disclosed herein, the human face and nose, given the changing curvature either from different people or same individual's movements, will cause the first support part to be pressed close to the nose wing, filling up gaps that may exist in the nose wing area between the eye and the light-shielding bodies, thus creating good light-blocking effect in this area.

The second support part will pressed close to the facial portion just under the eyes, filling up gaps that may exist in the under-the-eye area, thus creating a good light-blocking effect in this area.

With the embodiment disclosed herein, the light-shielding bodies, or the novel eye mask structure, affords an flexibility that adjusts to different facial curvature, or changing contour due to movements (while sleeping), and filled up gaps that may exist around the nose wing area and under the eye area, creating a close seal and better “darkness” setting that helps with quality sleep.

### DESCRIPTION OF THE DRAWINGS

The accompanying drawings exemplify the preferred embodiments of the invention. Together with the description, serve to explain the principles of the invention.

A brief description of the drawings is as follows:

FIG. 1 is a first embodiment of the light-shielding body structure of the present invention.

FIG. 2 is another embodiment of the light-shielding body structure.

FIG. 3 shows an embodiment of an eye mask structure of the present invention.

FIG. 4 is the exploded view of FIG. 3.

FIG. 5 shows an embodiment for the hard housing form of the outer shell.

FIG. 6 is an exploded view of the embodiment for the hard housing form of the outer shell.

FIG. 7 shows another embodiment of an eye mask structure with tie bands.

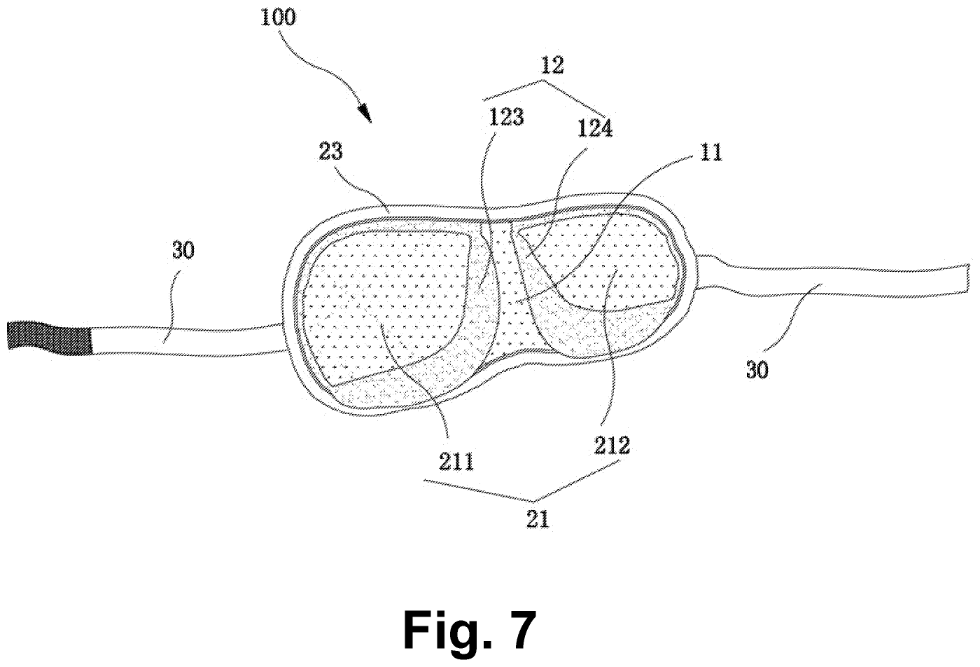

### DETAILED DESCRIPTION OF THE PREFERRED EMBODIMENT

In reference to the above technical background and summary of the invention, the preferred embodiments of the present invention, along with the illustrating drawings, are given herein.

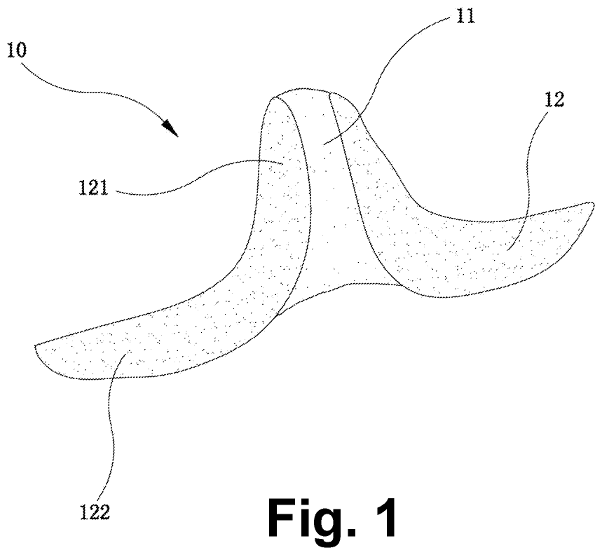

The light-shielding body structure 10, as shown in FIG. 1, of present invention primarily comprised of a base 11 and two light-shielding bodies 12 that can flexibly change shape when pressed upon and symmetrically placed on two sides of said base 10.

The two light-shielding bodies 12 are made to be arc-shaped and bent upwards relative to said base 10, forming an adjustment area that can be fitted to a human nose wing portion.

Each of the two light-shielding bodies 12 further consists of a first support part 121 that is meant to stay close to a human nose wing, and a second support part 122 that is meant to stay close to a human face, with the height of the first support part 121 gradually tapers down towards the second support part 122.

Each of the light-shielding bodies 12 further consists of a piece of cloth and a folded light-shielding sheet wrapped inside the cloth. The material for the cloth can be made from velvet, or similar material, to increase comfort level. The light-shielding sheet can be made from sponge. The folding of the light-shielding sheet creates a 3-D shape to provide a certain level of stiffness and resilience, even the general outside softness of being wrapped inside a cloth layer.

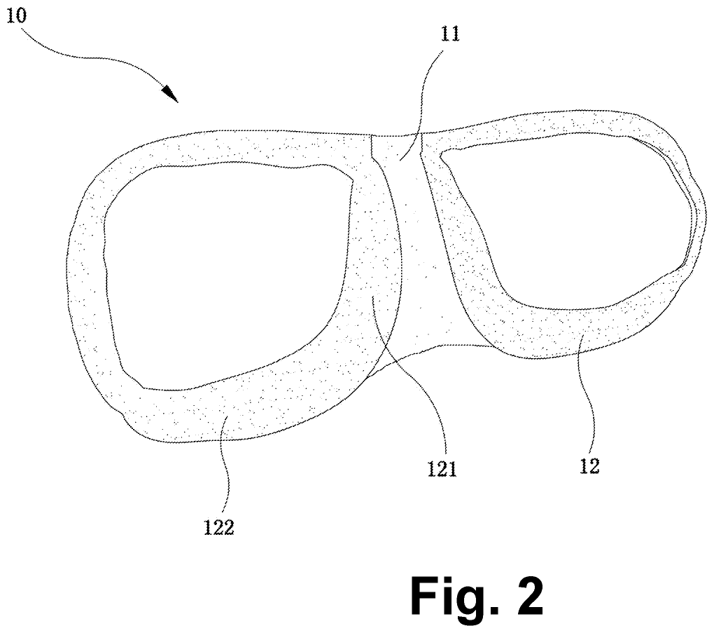

Each of the light-shielding body's 12 arc-shape is further made to form a ring shape; the two light-shielding bodies 12 are connected to the two sides of the base 11; the two ring-shaped light-shielding body 12 can be seen in FIG. 2.

In the case of application on a face mask, eye pad, eye patch or even a VR optical glasses, the two light-shielding bodies 12 of will be fitted around the eyes and nose, and given different human face, or given movements of an individual, the close-fitting will cause the first support part 121 to be pressed close to a human nose wing, filling up gaps that may exist in the nose wing area between the eye and the light-shielding bodies 12, thus creating good light-blocking effect in this area.

The second support part 122 will pressed close to the facial portion just under the eyes, filling up gaps that may exist in the under-the-eye area, thus creating a good light-blocking effect in this area.

A novel eye mask (or eye shade) structure 100 is formed by combining the light-shielding body structure 10 described above with a mask body 20 and two tie bands 30 on the two sides of the mask body 20.

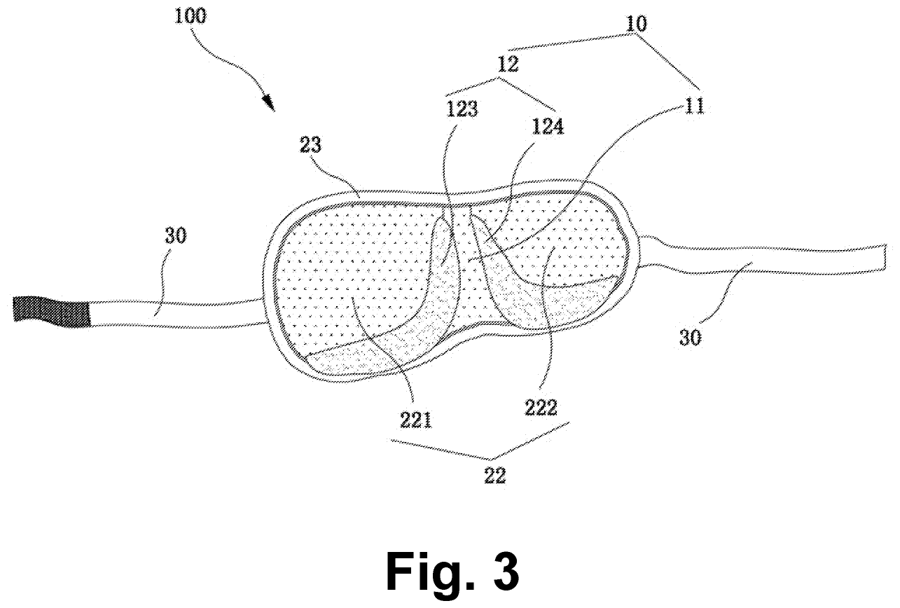

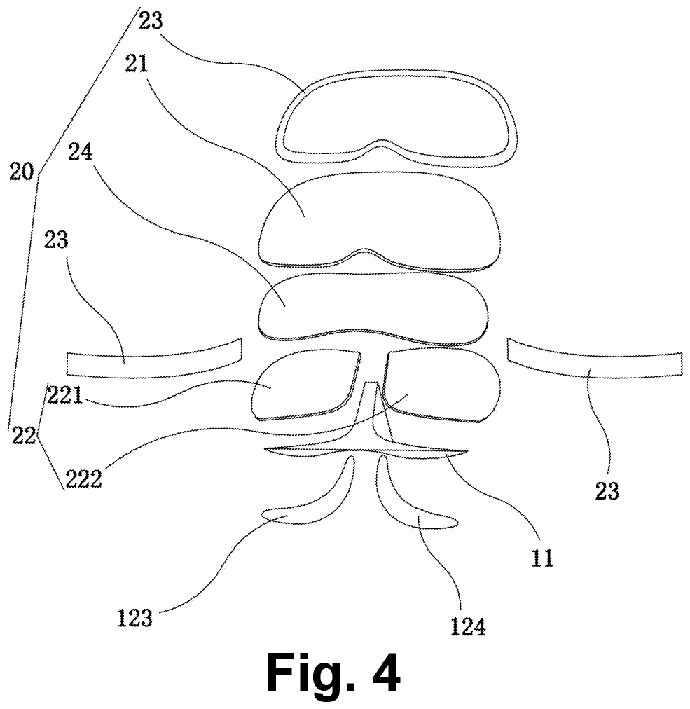

The novel eye mask structure 100, as shown in FIGS. 3 and 4, combining the light-shielding body structure 10 as described herein, further has a base 11 that is substantially the same as the shape of the mask body 20 and the base 11 is connected to inner portion of the mask body 20.

The novel eye mask structure 100 combining the light-shielding body structure 10 as described herein further has its base 11 contoured to the shape of a human nose, situated in a central part on the inside of the mask body 20, and the two light-shielding bodies 12 are connected to the two sides of the base 11 and the peripheral edge on the inside of the mask body 20 on the novel eye mask structure 100.

The novel eye mask structure 100 combining the light-shielding body structure 10 as described herein is further comprised of two light-shielding bodies 12 made up of a first light-shielding body 123 and a second light-shielding body 124, said first light-shielding body 123 is arc-shaped and is connected to the left side of the base 11 and to the lower-left side of the mask body's 20 inside surface, and said second light-shielding body 124 is arc-shaped and is connected to the right side of the base 11 and to the lower-right side of the mask body's 20 inside surface.

The novel eye mask structure 100 combining the light-shielding body structure 10 as described herein has its first light-shielding body 123 further connected to the left side and the upper-left part of the mask body's 20 inside surface and its second light-shielding body 124 further connected to the right side and upper-right part of the mask body's 20 inside surface.

The novel eye mask structure 100 combining the light-shielding body structure 10 as described herein further comprising an outer shell 21 and an inner contact piece 22. The light-shielding body 12 of the novel mask structure 100 is connected to the inner contact piece 22. Said inner contact piece 22 further consists of a first contact piece 221 and a second contact piece 222, so that the left side of the base 11 is connected to the first contact piece 221 via the first light-shielding body 123, and the right side of the base 11 is connected to the second contact piece 222 via the second light-shielding body 124.

When using the novel eye mask structure 100 of present invention, the mask body 20 will completely cover human eyes, with the light-shielding bodies, having flexible shape, filling up gaps around the eyes.

Specifically, the first support part 121 to be pressed close to a human nose wing, filling up gaps that may exist in the nose wing area between the eye and the light-shielding bodies 12, thus creating good light-blocking effect in this area.

The second support part 122 will pressed close to the facial portion just under the eyes, filling up gaps that may exist in the under-the-eye area, thus creating a good light-blocking effect in this area.

As such, for different people having different facial curvatures, or the same person having moving facial muscles during sleep, the light-shielding bodies 12 will create a snug-fit around the eyes, achieving good light-blocking effect.

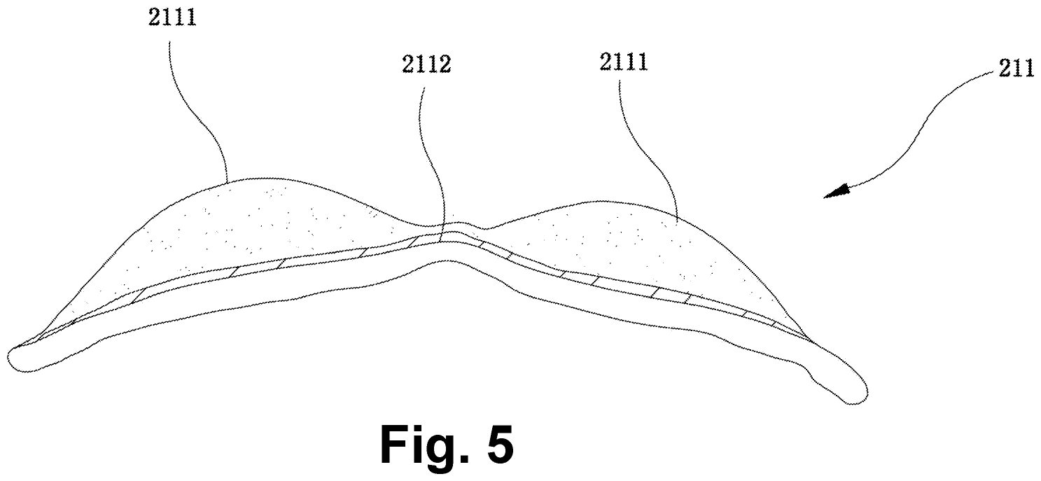

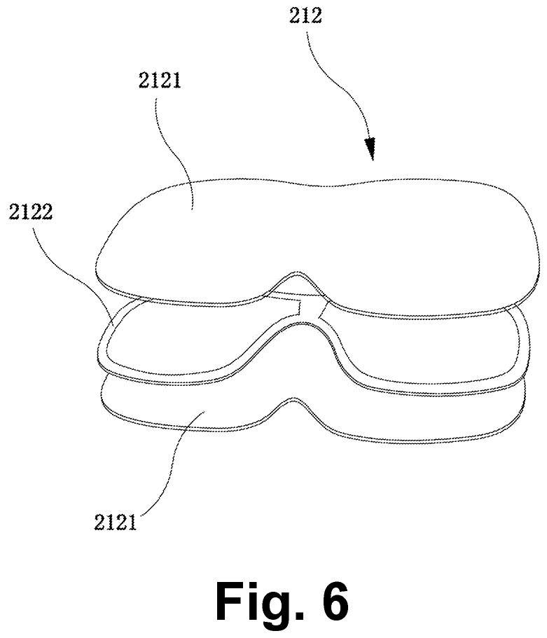

The novel eye mask structure 100 combining the light-shielding body structure 10 as described herein further has an outer shell 21 and an inner contact piece 22, as shown in FIGS. 4, 5, and 6.

The outer shell 21 and the inner contact piece 22 are connected via edge wrappings 23. The outer shell 21 may adopt a hard housing form, to prevent deformation of the mask body 20 during usage.

The hard housing form of outer shell 21 can be made by a one-piece molding press film 211, which is made from high-foaming material and multispandex wrapped in the surface layer of the high-foaming material. To prevent discomfort to the eyes due to excessive pressure, the part of the molding press film 211 corresponding to the eyes and nose wing will turn outward, forming a socket portion 2111 and a nose wing portion 2112.

As shown in FIG. 4, the hard housing of the outer shell 21 can be a composite hard housing 212, made up of a flexible layer 2121 and a molding frame sandwiched in the flexible layer 2121.

A middle layer 24 can be added to further increase the comfort level. The middle layer 24 is sandwiched between the outer shell 21 and the inner contact piece 22. The middle layer 24 can optionally be made up of pearl fabric, while the inner contact piece 22 can be made up of sponge and the multi spandex material wrapping over the sponge.

The above disclosure serves to explain the gist of the invention, with specific embodiments given. The disclosure is not meant to limit the scope of the invention; any equivalent implementation or disclosure, comporting with the spirit of the invention, should be deemed part of the invention.

1. A light-shielding body structure, comprising
a base; and
two light-shielding bodies that can flexibly change shape when pressed upon and ar symmetrically placed on two sides of said base, the two light-shielding bodies are made to be arc-shaped and bent upwards relative to said base, forming an adjustment area that can be fitted to a human nose wing portion.

2. The light-shielding body structure of claim 1, wherein each of the light-shielding bodies further consists of a first support part that is meant to stay close to a human nose wing, and a second support part that is meant to stay close to a human face, with the height of projection the first support part gradually tapers down towards the second support part.

3. The light-shielding body structure of claim 1, wherein each of the light-shielding bodies consists of a piece of cloth and a folded light-shielding sheet wrapped inside the cloth.

4. The light-shielding body structure of claim 1 or 2 or 3, wherein the light-shielding bodies's arc-shape further form a ring-shape and are connected to the two sides of the base.

5. A light-shielding body structure of claim 1, or 2 or 3, further comprising a
a mask body and two tie bands on the two sides of the mask body forming an novel eye mask structure.

6. A light-shielding body structure of claim 5, wherein the shape of the base is substantially the same as the shape of the mask body and the base is connected to inner portion of the mask body on the novel eye mask structure.

7. The light-shielding body structure of claim 5, wherein the shape of the base is contoured to the shape of a human nose, situated in a central part on the inside of the mask body, and the two light-shielding bodies are connected to the two sides of the base and the peripheral edge on the inside of the mask body on the novel eye mask structure.

8. The light-shielding body structure of claim 7, wherein the two light-shielding bodies consist of a first light-shielding body and a second light-shielding body, said first light-shielding body is arc-shaped and is connected to the left side of the base and to the lower-left side of the mask body's inside surface, and said second light-shielding body is arc-shaped and is connected to the right side of the base and to the lower-right side of the mask body's inside surface.

9. The light-shielding body structure of claim 8, wherein said first light-shielding body is further connected to the left side and the upper-left part of the mask body's inside surface and said second light-shielding body is further connected to the right side and upper-right part of the mask body's inside surface.

10. The light-shielding body structure of claim 8 or 9, further comprising
an outer shell; and
an inner contact piece to form a novel eye mask structure
wherein the light-shielding body is connected to the inner contact piece, said inner contact piece further consists of a first contact piece and a second contact piece, so that the left side of the base is connected to the first contact piece via the first light-shielding body, and the right side of the base is connected to the second contact piece via the second light-shielding body.

11. The light-shielding body structure of claim 5 wherein the outer shell of the novel eye mask is further made of a composite hard housing consisting of a flexible layer and a molding frame sandwiched in the flexible layer.
